(19) United States
(12) Patent Application Publication
(10) Pub. No.: US 2026/0069153 A1
(43) Pub. Date: Mar. 12, 2026

(54) FLOW PATH PLATE UNIT AND BLOOD PRESSURE MEASUREMENT DEVICE

(71) Applicant: OMRON HEALTHCARE Co., Ltd., Kyoto (JP)

(72) Inventors: Takaaki OKANISHI, Kyoto (JP); Yoshihide TOKKO, Kyoto (JP); Misaki TERUYAMA, Kyoto (JP)

(21) Appl. No.: 19/389,921

(22) Filed: Nov. 14, 2025

Related U.S. Application Data
(63) Continuation of application No. PCT/JP2024/002951, filed on Jan. 30, 2024.

(30) Foreign Application Priority Data
Jul. 21, 2023 (JP) 2023-118850

(51) Int. Cl. A61B 5/021; A61B 5/00

(52) U.S. Cl. CPC A61B 5/02141; A61B 5/681

(57) ABSTRACT

A flow path plate unit includes a flow path for fluid and connecting portions for a pump and a cuff. The unit comprises: a first plate member having at least the connecting portion with the pump; a second plate member having at least the connecting portion with the cuff; a thin film sheet disposed between the first and second plate members and provided with one or more orifices; a first thick film sheet disposed between the first plate member and the thin film sheet, thicker than the thin film sheet, and including a flow path communicating an orifice of the thin film sheet with the pump-connecting portion; and a second thick film sheet disposed between the second plate member and the thin film sheet, thicker than the thin film sheet, and including a flow path communicating an orifice of the thin film sheet with the cuff-connecting portion.

### CROSS-REFERENCE TO RELATED APPLICATIONS

This application is the U.S. national stage application filed pursuant to 35 U.S.C. 365(c) and 120 as a continuation of International Patent Application No. PCT/JP2024/002951, filed Jan. 30, 2024, which application claims priority to Japanese Patent Application No. 2023-118850, filed Jul. 21, 2023, which applications are incorporated herein by reference in their entireties.

### FIELD

The present invention belongs to the technical field of healthcare, and particularly relates to a blood pressure measurement device and a flow path plate unit used therein.

### BACKGROUND

A device commonly known as a blood pressure measurement device uses the oscillometric method, in which a cuff wound around an upper arm, wrist, or the like of a living body is inflated and deflated, and the pressure of the cuff is detected by a pressure sensor to detect a pulse wave and measure blood pressure, for example. In recent years, it has become common for individuals to measure information relating to their own body and health, such as blood pressure values (hereinafter also referred to as biometric information), on a daily basis using a measuring apparatus, and to utilize the measurement results for health management. Accordingly, there has been an increasing demand for apparatuses with an emphasis on portability, and many portable measuring devices have been proposed (for example, Patent Document 1: JP2018-143557 A, and the like).

In such portable devices, a unit in which a flow path is formed inside a plate-like member is used. In wearable devices, not limited to blood pressure measurement devices, it is necessary to achieve miniaturization and weight reduction to improve wearability as much as possible while at the same time properly performing the supply and discharge of fluid for accurate blood pressure measurement, which requires appropriate (accurate) setting of the flow path resistance within the flow path.

As an invention relating to such a flow path unit, an invention of a microchannel chip used in an immunochromatography method is known (Patent Document 2: JP2003-114229 A) although it is different from the field of blood pressure measurement devices. In the invention disclosed in Patent Document 2, an appropriate flow path resistance is achieved by laminating a plurality of substrates and film-like members, providing flow path patterns in each layer, joining the layers to three-dimensionally form flow paths, and changing the width of the flow path provided in the horizontal plane direction in the film-like member.

### SUMMARY

### Technical Problem

In the invention disclosed in Patent Document 2, since the flow path resistance is provided by the flow path disposed in the horizontal plane (XY plane) direction of the film-like member, there is a problem that the flow path resistance error due to the manufacturing method is large. The flow path resistance error arises from dimensional errors of the flow path itself, gaps or protrusions of bonding members occurring when joining sheets, variations in flow path length due to misalignment of the sheets, and the like. With the configuration of the flow path plate disclosed in Patent Document 2, it is technically difficult to reduce these errors, and even if realized, an increase in cost is expected.

In view of the above-described problems, an object of the present invention is to provide a technique of cost-effectively achieving a fluid circuit with small flow path resistance error.

### Solution to Problem

To solve the above-mentioned problems, the present invention adopts the following configurations. A flow path plate unit in which a flow path for fluid is formed, and connecting portions with a pump and a cuff are provided, the flow path plate unit including: a first plate member in which at least the connecting portion with the pump is formed; a second plate member in which at least the connecting portion with the cuff is formed; a thin film sheet disposed between the first plate member and the second plate member and provided with one or more orifices; a first thick film sheet disposed between the first plate member and the thin film sheet and formed to have a thickness greater than that of the thin film sheet, the first thick film sheet including a flow path through which a corresponding one of the orifices in the thin film sheet and the connecting portion with the pump in the first plate member communicate with each other; and a second thick film sheet disposed between the second plate member and the thin film sheet and formed to have a thickness greater than that of the thin film sheet, the second thick film sheet including a flow path through which a corresponding one of the orifices in the thin film sheet and the connecting portion with the cuff in the second plate member communicate with each other.

With such a configuration, the flow path resistance for controlling the flow rate (flow velocity) can be designed as a simple hole (orifice). In addition, by separating the thin film sheet layer including the orifices (a flow path resistance for control) from other layers, the flow path resistance can be managed based on the performance of the orifice portion, and thus the influence on the performances of the pump and the valve can be minimized. In this manner, a flow path plate unit with small flow path resistance error can be cost-effectively achieved.

The first plate member, the first thick film sheet, the thin film sheet, the second thick film sheet, and the second plate member may be joined to each other by a bonding member. Further, the first thick film sheet and the second thick film sheet may be double-sided tapes, the first thick film sheet may be joined to the first plate member on one surface, and joined to the thin film sheet on the other surface, and the second thick film sheet may be joined to the second plate member on one surface, and joined to the thin film sheet on the other surface.

With such a configuration, the first thick film sheet and the second thick film sheet themselves can also serve as bonding members, and thus thickness error of the flow path plate unit can be easily suppressed. In addition, each of the first plate member, the first thick film sheet, the thin film sheet, the second thick film sheet, and the second plate member can be easily bonded, thereby contributing to simplification of assembly.

In addition, the thin film sheet may be a metal sheet in which the orifices are provided, and is formed in a shape with an area smaller than each of the first plate member, the second plate member, the first thick film sheet, and the second thick film sheet. With such a configuration, in comparison with the case where the thin film sheet is formed as a sheet having the same area as that of other members, the manufacturing cost of the flow path plate unit can be reduced. Note that in the case where the thin film sheet is formed of a metal, methods such as laser processing and electroforming can be used in accordance with the size and shape of the thin film sheet.

The flow path plate unit further includes a film member disposed between the first plate member and the second plate member, and having substantially the same thickness as that of the thin film sheet, the film member including an opening portion having a shape within which the thin film sheet is accommodated in plan view, in which the thin film sheet may be disposed at the opening portion of the film member. With such a configuration, even in the case where the thin film sheet is formed with an area smaller than those of the first plate member, the second plate member, the first thick film sheet, and the second thick film sheet, the overall thickness of the layer where the thin film sheet is disposed can be substantially the same as that of the thin film sheet. In this manner, the joining state of the adjacently disposed members such as the first and second thick film sheets can be stabilized, and the risk of air leakage can be reduced.

In addition, the first thick film sheet and the second thick film sheet may be double-sided tapes, and the flow path plate unit may further include: a first film sheet disposed between the first thick film sheet and the thin film sheet, a first bonding sheet disposed between the first film sheet and the thin film sheet, a second film sheet disposed between the second thick film sheet and the thin film sheet, and a second bonding sheet disposed between the second film sheet and the thin film sheet.

With such a configuration, complex flow path routing can be achieved, and the area of the flow path plate unit can be reduced. In addition, even in the configuration in which the thin film sheet includes the orifice formation portion and the film portion, the constriction of the flow path can be prevented.

In addition, the orifices may be formed to have a tapered shape that opens on a side into which the fluid flows. With such a configuration, the pressure dependence of the flow path resistance resulting from fluid viscosity can be reduced, thereby enabling an increased flow rate to the cuff from low pressure during pressurization (cuff inflation), and preventing residual air during exhaust at low pressure.

In addition, in each of the first plate member, the first thick film sheet, the thin film sheet, the second thick film sheet, and the second plate member, a notch or a hole may be formed for positioning the first plate member, the first thick film sheet, the thin film sheet, the second thick film sheet, and the second plate member when they are to be joined to each other. In this manner, positioning error of each layer can be reduced.

In addition, the present invention is also applicable as a blood pressure measurement device including the flow path plate unit.

It should be noted that each of the above configurations and processes may be combined with one another to constitute the present invention, as long as no technical inconsistency arises.

### Advantageous Effects of Invention

According to the present invention, it is possible to provide a technique of cost-effectively achieving a fluid circuit with small flow path resistance error.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a perspective view schematically illustrating an external appearance of a blood pressure measurement device according to an embodiment of the present invention.

FIG. 2 is a side view schematically illustrating the blood pressure measurement device according to the embodiment.

FIG. 3 is an explanatory diagram illustrating a positional relationship when the blood pressure measurement device according to the embodiment is worn on the wrist.

FIG. 4 is a schematic sectional view of the blood pressure measurement device according to the embodiment as viewed from a lateral side.

FIG. 5 is a perspective view illustrating an external appearance of the flow path plate unit according to the embodiment.

FIG. 6 is a block diagram schematically illustrating a functional configuration of the blood pressure measurement device according to the embodiment, and an air flow path within the flow path plate unit.

FIG. 7 is an exploded perspective view of the flow path plate unit according to the embodiment.

FIG. 8 is an exploded perspective view of the flow path plate unit according to the embodiment.

FIG. 9 is an explanatory diagram illustrating a structure of an orifice portion in the flow path plate unit according to the embodiment.

FIG. 10 is an exploded perspective view of a flow path plate unit according to a modification.

### DESCRIPTION OF EMBODIMENTS

### First Embodiment

An embodiment of the present invention is specifically described below with reference to the drawings. Note that unless otherwise specified, the dimensions, materials, shapes, relative arrangements, and the like of the components described in the following embodiments are not intended to limit the scope of the present invention to those alone.

### Device Configuration

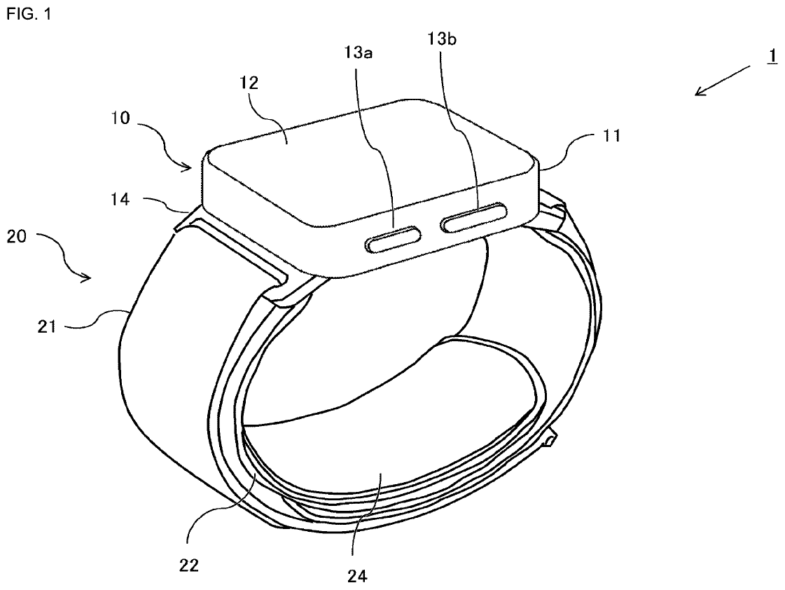

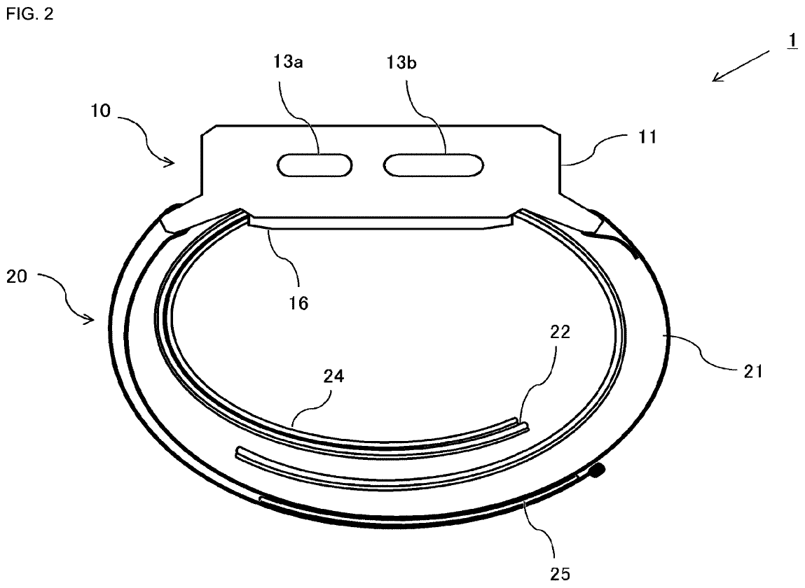

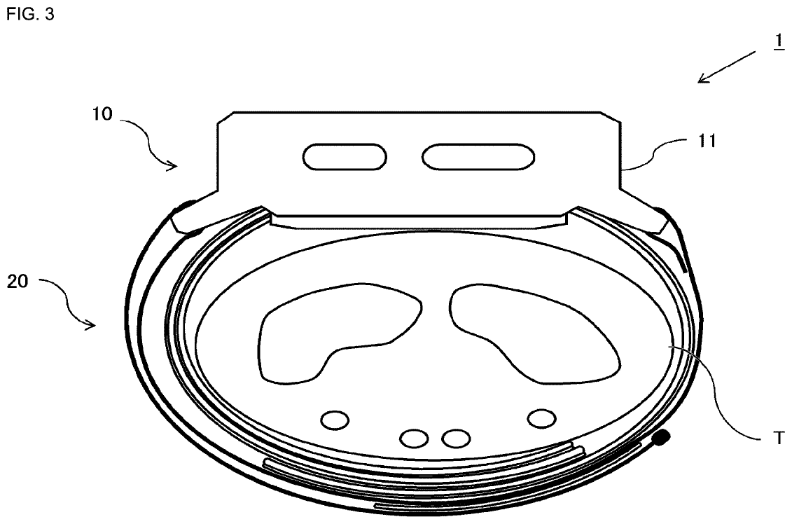

FIG. 1 is a perspective view schematically illustrating an external appearance of a configuration of a blood pressure measurement device 1 according to the present embodiment. In addition, FIG. 2 is a side view schematically illustrating a configuration of the blood pressure measurement device 1 according to the present embodiment. As illustrated in FIGS. 1 and 2, the blood pressure measurement device 1 is generally a wristwatch-type wearable device including a main body portion 10 and a belt portion 20, and is capable of measuring a blood pressure value while being worn on a wrist T of a human body. FIG. 3 illustrates a positional relationship between the wrist and the components of the blood pressure measurement device 1 when the blood pressure measurement device 1 according to the present embodiment is worn on the wrist T.

As illustrated in FIGS. 1 and 2, the main body portion 10 includes a main body case 11 and a cuff cover 16 described later. The main body case 11 is provided with a display 12 (such as an organic EL display), operation buttons 13a and 13b, a lug 14 and the like. Note that in the present embodiment, the side on which the display 12 is formed is the front surface of the main body case 11, and the side on which the cuff cover 16 is provided is the bottom portion of the main body case 11. In addition, in the following description, the front surface side of the main body case 11 may be referred to as the upper side, and the bottom portion side of the main body case 11 may be referred to as the lower side.

The belt portion 20 includes, in addition to a belt 21 and a surface fastener 25 for fixing the blood pressure measurement device 1 to the wrist T, a pressing cuff 22 for compressing an artery in the wrist T and a sensing cuff 24 for detecting a pulse wave. Note that the connecting portion of the pressing cuff 22 and the sensing cuff 24, and the main body case 11 is covered with the cuff cover 16. The cuff cover 16 has a function of protecting the connecting portion of the pressing cuff 22 and the sensing cuff 24, and the main body case 11, and fixing each cuff to the main body case 11.

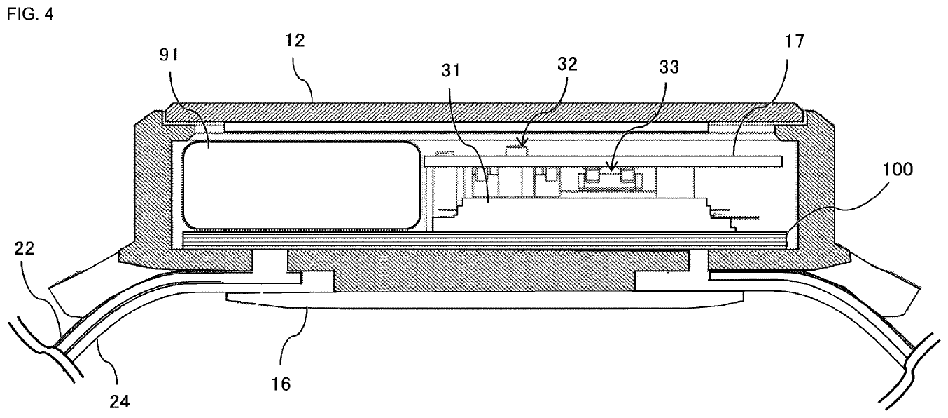

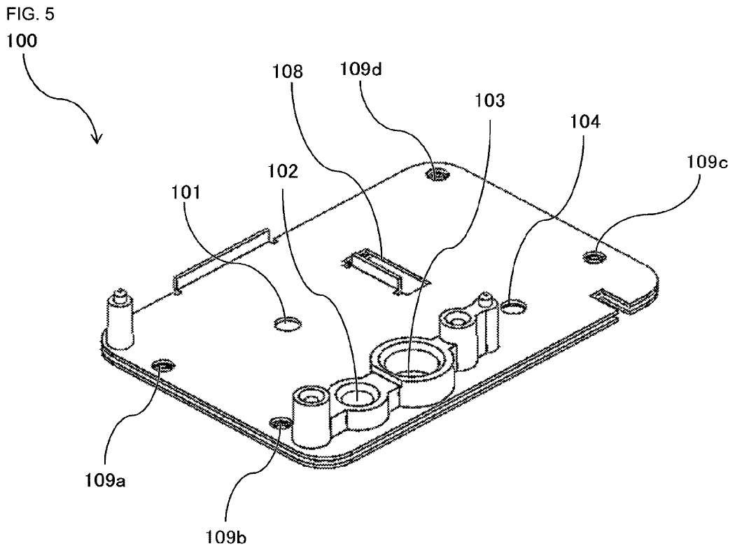

FIG. 4 is a schematic sectional view of the main body case 11 as viewed from a lateral side, and an overview of an internal configuration of the main body case 11 of the blood pressure measurement device 1. As illustrated in FIG. 4, the main body case 11 houses inside a rechargeable battery 91, a control board 17, a pump 31, a valve 32, a pressure sensor 33, a flow path plate unit 100 and the like. FIG. 5 is a perspective view schematically illustrating an external appearance of the flow path plate unit 100.

For example, the rechargeable battery 91 may be a general-purpose secondary battery such as a lithium ion battery, and can be repeatedly charged by receiving power supply through a charging terminal not illustrated in the drawing. In addition, on the control board 17, a processor such as a central processing unit (CPU) not illustrated in the drawing, a memory such as a random access memory (RAM) and the like are mounted.

The pump 31 is, for example, a piezoelectric pump, and electrically connected to the control board 17. The pump 31 compresses air and supplies it to the pressing cuff 22 and the sensing cuff 24 through the flow path plate unit 100, for example. As described later, the valve 32 is connected to a valve connecting portion 102 of the flow path plate unit 100, and releases the air supplied to the pressing cuff 22 to the atmosphere. The valve 32 is electrically connected to the control board 17, and opened and closed by the control of the processor. The pressure sensor 33 is connected to a sensor connecting portion 103 of the flow path plate unit 100, and detects the pressure of the sensing cuff 24 via the air flowing through the flow path formed within the flow path plate unit 100. Specifically, the pressure sensor 33 is electrically connected to the control board 17, and converts the detected pressure into an electric signal and outputs it to the processor.

The pressing cuff 22, which is connected to a pressing cuff connecting portion 106 (not illustrated in FIG. 5) disposed on the lower side of the flow path plate unit 100, inflates with the air sent from the pump 31 to tighten the wrist T at the wearing site and apply external pressure to the artery in the wrist T. In addition, the sensing cuff 24 is a fluid bag for detecting the pressure applied on the site compressed by the pressing cuff 22, and is connected to a sensing cuff connecting portion 107 (not illustrated in FIG. 5) of the flow path plate unit 100. By detecting the inner pressure of the sensing cuff 24 with the pressure sensor 33 in the state where a small amount of air is contained in the sensing cuff 24, the pressure applied on the compressed site is measured.

### Functional Configuration of Device

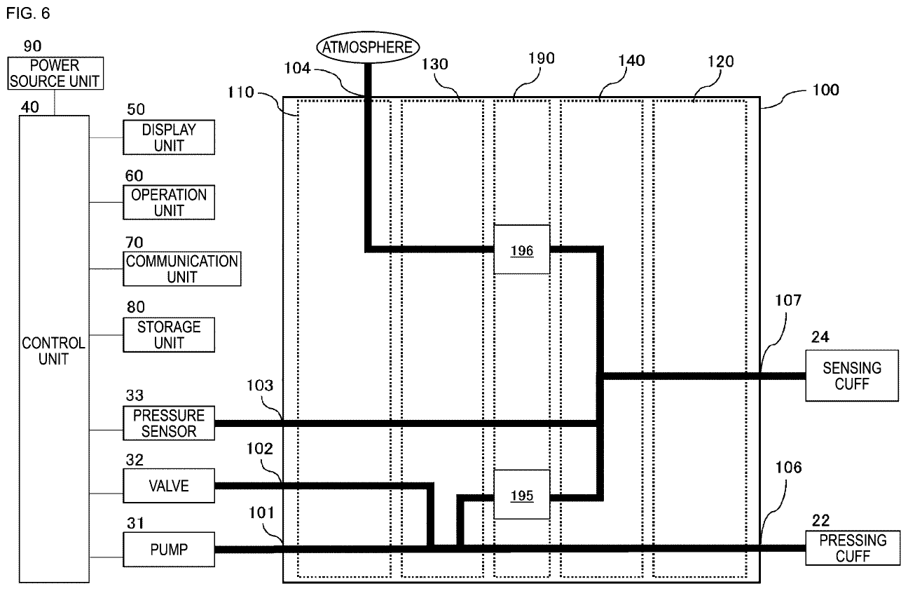

Next, a functional configuration of the blood pressure measurement device 1 is described. FIG. 6 is a block diagram schematically illustrating a functional configuration of the blood pressure measurement device 1, and a flow path of air. Note that the thick solid line in FIG. 6 schematically indicates a flow path of air flowing through each layer in the flow path plate unit 100. As illustrated in FIG. 6, the blood pressure measurement device 1 according to the present embodiment includes functional units, namely a control unit 40, a display unit 50, an operation unit 60, a communication unit 70, a storage unit 80, and a power source unit 90. These functional units are implemented when the processor of the control board 17 controls each configuration of the blood pressure measurement device 1 by reading a program from the memory and executing it, for example.

The control unit 40 includes a processor (not illustrated in the drawing) of the control board 17, and manages the overall control of the blood pressure measurement device 1. Specifically, for example, it controls the pump 31, the valve 32, the pressure sensor 33 and the like, and measures the blood pressure by a so-called oscillometric method. The blood pressure measurement using the oscillometric method is a known technique, and therefore the description thereof is omitted.

The display unit 50 includes the display 12, and displays various information such as measurement results of blood pressure and a menu screen. The operation unit 60 includes the operation buttons 13a and 13b, and receives inputting operations of the user through them.

The communication unit 70 includes an interface for wired or wireless communication (not illustrated in the drawing), and performs information communication with an external device such as an information processing terminal using BLE communication, for example. More specifically, it transmits information on the measured blood pressure value, pulse and the like to an external device, and receives a program for software updates and the like from the external device and sends it to the control unit 40. The external device is an information processing terminal such as a smartphone, a tablet terminal, a personal computer, and a smart watch, for example.

The storage unit 80 includes a main storage device such as a RAM, and stores various information such as an application program and measured biometric information. In addition, it may include a long-term storage medium such as a flash memory in addition to the RAM, for example. The power source unit 90 includes the rechargeable battery 91, and functions as a power supply source to each unit making up the blood pressure measurement device 1.

### Configuration of Flow Path Plate Unit

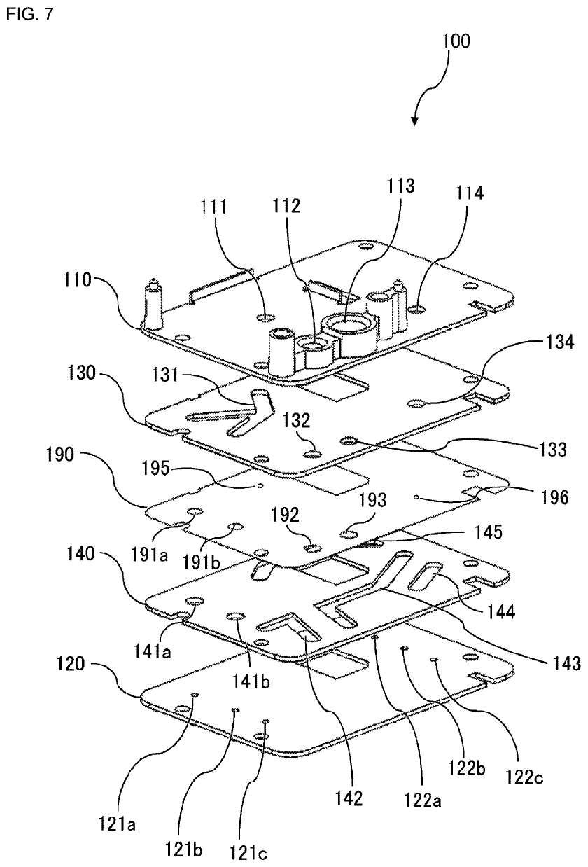

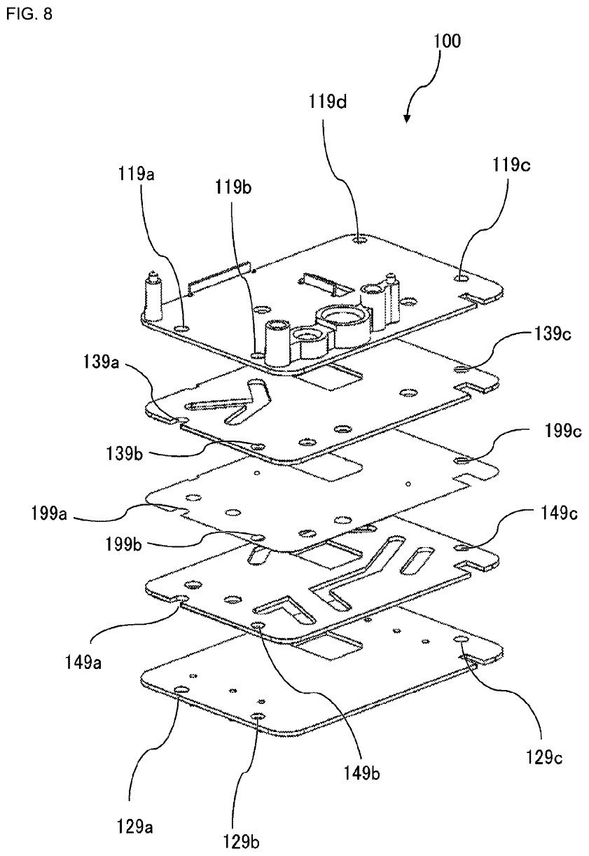

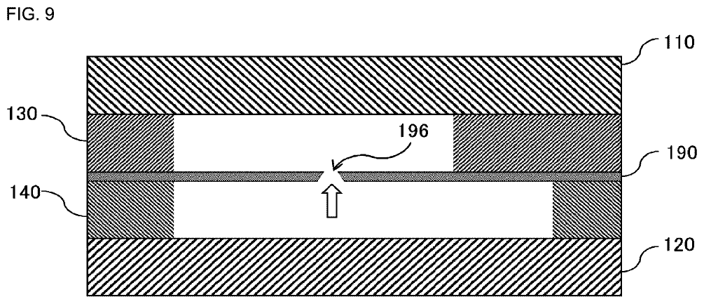

Now, with reference to FIGS. 5 to 9, details of a configuration of the flow path plate unit 100 and air flow inside the blood pressure measurement device 1 are described. FIGS. 7 and 8 are exploded perspective views of the flow path plate unit 100, and FIG. 9 is a schematic sectional view of the flow path plate unit 100 illustrating a structure of an orifice (second orifice 196) described later.

As illustrated in FIG. 5, the flow path plate unit 100 is formed in a substantially rectangular plate shape having dimensions of approximately 40 mm in length, 25 mm in width, and 1 mm in thickness, and provided with a boss and a nozzle for connection with other configurations of the blood pressure measurement device 1, for example. Openings such as a pump connecting portion 101 connected to the pump 31, the valve connecting portion 102 connected to the valve 32, the sensor connecting portion 103 connected to the pressure sensor 33, and an atmospheric release port 104 are provided at the upper side surface of the flow path plate unit 100. In addition, an opening portion 108 for avoiding interference with the components (not illustrated in the drawing) housed inside the main body case 11 is provided so as to extend through the vicinity of the center portion of the flow path plate unit 100 in plan view. In addition, screw insertion holes 109a, 109b, 109c and 109d for insertion of screws for fixing with the main body case 11 are provided in the vicinity of the four corners of the rectangular. Further, although not illustrated in FIG. 5, the openings of the pressing cuff connecting portion 106 connected to the pressing cuff 22 and the sensing cuff connecting portion 107 connected to the sensing cuff are provided on the lower side of the flow path plate unit 100.

In addition, as illustrated in FIGS. 7 and 8, the flow path plate unit 100 is formed with a stack of five sheet members, namely, a first metal plate 110, a first thick film sheet 130, a thin film sheet 190, a second thick film sheet 140, and a second metal plate 120, which are joined in this order from the upper side. More specifically, the first thick film sheet 130 and the second thick film sheet 140 are double-sided tapes, and the first thick film sheet 130 and the first metal plate 110 and the thin film sheet 190, and the second thick film sheet 140 and the second metal plate 120 and the thin film sheet 190 are joined together.

The first metal plate 110 is formed of a metal material such as stainless-steel, and provided with a pump connection hole 111 making up the pump connecting portion 101, a valve connection hole 112 making up the valve connecting portion 102, a sensor connection hole 113 making up the sensor connecting portion 103, and the atmospheric connection hole 114 making up the atmospheric release port 104. In addition, as illustrated in FIG. 8, in the vicinity of the four corners of the first metal plate 110, positioning openings 119a, 119b, 119c and 119d functioning as positioning portions for joining each layer and making up the screw insertion holes 109a, 109b, 109c and 109d are provided. The first metal plate 110 has a thickness of, for example, 0.35 mm.

In addition, the first thick film sheet 130 is, for example, a double-sided tape including a base material such as an acrylic foam material. In the first thick film sheet 130, a first communication flow path 131 as an opening portion that branches air sent from the pump 31 and connects to the flow path of the thin film sheet 190, a valve connection hole 132 making up a flow path of air to the valve 32, a sensor connection hole 133 making up a flow path of air to the pressure sensor 33, and the atmospheric connection hole 134 making up a flow path of air to the atmospheric release port 104 are formed. In addition, as illustrated in FIG. 8, in the vicinity of the four corners of the first thick film sheet 130, four positioning openings (139a, 139b, 139c, and one not illustrated in the drawing) functioning as positioning portions for joining each layer and making up the screw insertion holes 109a, 109b, 109c and 109d are provided. The first thick film sheet 130 has a thickness of, for example, 0.15 mm.

The thin film sheet 190 is formed of a metal such as nickel, and in the thin film sheet 190, pressing cuff connection holes 191a and 191b making up a flow path of air to the pressing cuff 22, a valve connection hole 192 making up a flow path of air to the valve 32, a sensor connection hole 193 making up a flow path of air to the pressure sensor 33, and a first orifice 195 and the second orifice 196 as openings functioning as flow rate resistances are formed by laser processing or the like, for example.

The first orifice 195 is a connection hole making up an air flow path from the first communication flow path 131 of the first thick film sheet 130 to a fifth communication flow path 145 of the second thick film sheet 140 described later. Since the hole diameter of the first orifice 195 is smaller than the width of the first communication flow path 131, the first orifice 195 serves as a flow rate resistance, and the air flow rate in the flow path downstream of the first orifice 195 is reduced compared to the upstream portion. The second orifice 196 is a connection hole making up an air flow path from a fourth communication flow path 144 of the second thick film sheet 140 described later to the atmospheric connection hole 134 of the first thick film sheet 130. Since the hole diameter of the second orifice 196 is smaller than the width of the fourth communication flow path 144, the second orifice 196 serves as a flow rate resistance, and the air flow rate in the flow path downstream of the second orifice 196 is reduced compared to the upstream portion. The first thick film sheet 130 and the second thick film sheet 140 are formed with a larger thickness than that of the thin film sheet 190. In this manner, the fluid resistance value of the flow paths formed in the first thick film sheet 130 and the second thick film sheet 140 is negligibly smaller than the fluid resistance value of the first orifice 195 and the second orifice 196, and thus the fluid resistance value can be accurately set by the hole diameters of the first orifice 195 and the second orifice 196.

Here, the orifice may be a cylindrical conduit, or a tapered conduit that opens on the side into which air flows. For example, as illustrated in FIG. 9, the second orifice 196 is formed in a tapered shape that opens on the second thick film sheet 140 side. The white arrow illustrated in FIG. 9 indicates the air flow direction. By forming the orifice in such a tapered shape, it can be more easily processed than a cylindrical shape, and the flow path resistance value can be set by the hole diameter of the orifice on the small diameter side. The diameter of the tapered shape may be approximately 50 μm on the large diameter side, and approximately 30 μm on the small diameter side, for example. By forming the tapered orifice in the sheet having a small thickness, the pressure dependence of the flow path resistance resulting from fluid viscosity can be reduced, thereby enabling an increased flow rate to the cuff from low pressure during pressurization, and preventing residual air during exhaust at low pressure.

In addition, as illustrated in FIG. 8, in the vicinity of the four corners of the thin film sheet 190, four positioning openings (199a, 199b, 199c, and one not illustrated in the drawing) functioning as positioning portions for joining each layer and making up the screw insertion holes 109a, 109b, 109c and 109d are provided. The thin film sheet 190 has a thickness of, for example, 0.05 mm.

The second thick film sheet 140 is, for example, a double-sided tape including a base material such as an acrylic foam material. In the second thick film sheet 140, pressing cuff connection holes 141a and 141b that form a flow path of air to the pressing cuff 22 are formed. In addition, in the second thick film sheet 140, a second communication flow path 142 that connects a pressing cuff connection hole 121c of the second metal plate 120 described later and the valve connection hole 192 of the thin film sheet 190 and serves as a flow path of air to be exhausted from the pressing cuff 22 is formed. In addition, in the second thick film sheet 140, a third communication flow path 143 that connects a sensing cuff connection hole 122b of the second metal plate 120 described later and the sensor connection hole 193 of the thin film sheet 190 and makes up a flow path of air from the sensing cuff 24 to the pressure sensor 33 are formed. In addition, in the second thick film sheet 140, the fourth communication flow path 144 that connects a sensing cuff connection hole 122c of the second metal plate 120 described later and the second orifice 196 of the thin film sheet 190 is formed. In addition, in the second thick film sheet 140, the fifth communication flow path 145 that connects the first orifice 195 of the thin film sheet 190 and a sensing cuff connection hole 122a of the second metal plate 120 described later and serves as a flow path of air to the sensing cuff 24 is formed.

In addition, as illustrated in FIG. 8, in the vicinity of the four corners of the second thick film sheet 140, four positioning openings (149a, 149b, 149c, and one not illustrated in the drawing) functioning as positioning portions for joining each layer and making up the screw insertion holes 109a, 109b, 109c and 109d are provided. The second thick film sheet 140 has a thickness of, for example, 0.15 mm.

The second metal plate 120 is formed of a metal material such as stainless steel. In the vicinity of one end of the second metal plate 120 in the longitudinal direction, pressing cuff connection holes 121a and 121b making up a flow path of air that flows into the pressing cuff 22, and a pressing cuff connection hole 121c making up a flow path of air to be discharged from the pressing cuff 22 are formed. In addition, in the vicinity of the other end in the longitudinal direction, the sensing cuff connection hole 122a making up a flow path of air that flows into the sensing cuff 24, and sensing cuff connection holes 122b and 122c making up a flow path of air to be discharged from the sensing cuff 24 are formed. Note that the pressing cuff connection holes 121a, 121b and 121c make up the pressing cuff connecting portion 106, and the sensing cuff connection holes 122a, 122b, 122c make up the sensing cuff connecting portion 107.

In addition, as illustrated in FIG. 8, in the vicinity of the four corners of the second metal plate 120, four positioning openings (129a, 129b, 129c, and one not illustrated in the drawing) functioning as positioning portions for joining each layer and making up the screw insertion holes 109a, 109b, 109c and 109d are provided. The second metal plate 120 has a thickness of, for example, 0.35 mm.

### Effects of Present Embodiment

With the above-described configuration of the flow path plate unit 100, the flow rate resistance of the flow path can be adjusted by the first orifice 195 and the second orifice 196 formed in the thin film sheet 190, thereby reducing resistance errors in the flow path, and cost-effectively achieving a flow path with smaller resistance errors. In addition, the flow path plate unit 100 is formed with two metal plates and thin film sheets joined with double-sided tapes in which flow paths are formed, and the flow paths can be appropriately sealed.

### Modifications

Note that in the above-mentioned embodiment, the flow path plate unit 100 is composed of five sheet members, but such a configuration is not limitative. A flow path plate unit 200 according to a modification is described below. Note that in the following description, components common to those of the blood pressure measurement device 1 of the first embodiment are denoted with the same reference symbols, and the description thereof is omitted.

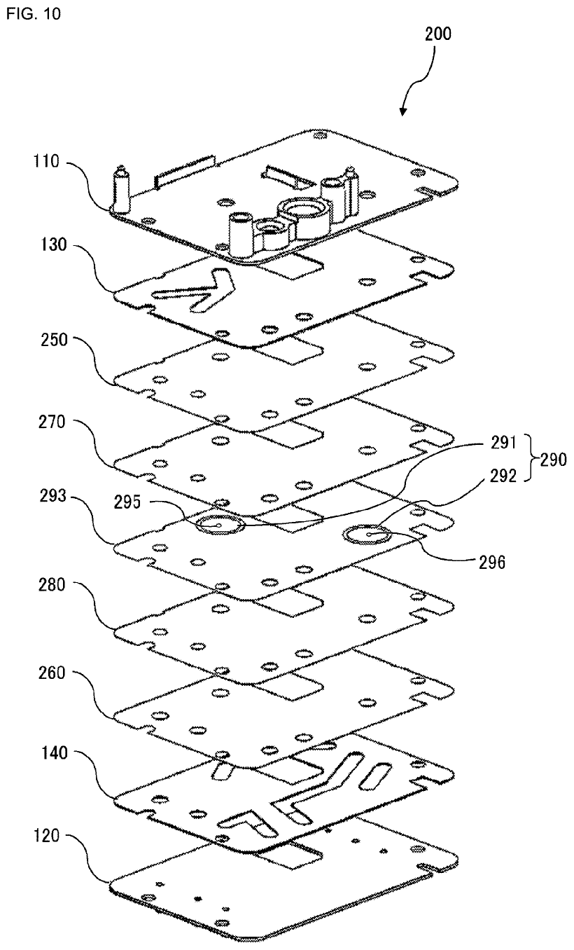

FIG. 10 is an exploded perspective view of the flow path plate unit 200. Note that the external appearance of the flow path plate unit 200 is substantially the same as that of the flow path plate unit 100 as illustrated in FIG. 5. In addition, the air flow between the pump 31, the pressing cuff 22, the sensing cuff 24, the valve 32, the pressure sensor 33, and the atmospheric release port 104 through the flow path plate unit 200 is also the same as that of the first embodiment. Specifically, the air flow within the flow path plate unit 200 is the same as that of the flow path plate unit 100.

As illustrated in FIG. 10, as with the flow path plate unit 100, the flow path plate unit 200 includes the first metal plate 110, the first thick film sheet 130, a thin film sheet 290, the second thick film sheet 140, and the second metal plate 120 as its components. Further, other than these sheet members, a buffer film 250 and a double-sided tape 270 are disposed between the first thick film sheet 130 and the thin film sheet 290, and a buffer film 260 and a double-sided tape 280 are disposed between the second thick film sheet 140 and the thin film sheet 290.

Each of the buffer films 250 and 260 is a resin (PET) film, and has a thickness of, for example, 0.025 mm. Each of the double-sided tapes 270 and 280 includes a base material such as an acrylic foam material, and has a thickness of, for example, 0.03 mm.

In addition, the buffer films 250 and 260 and the double-sided tapes 270 and 280 are provided with the same shape and the same opening in plan view. More specifically, there are an opening making up an air flow path flowing from the pump 31 to the pressing cuff 22, an opening making up a flow path connecting the first communication flow path 131 of the first thick film sheet 130 and the fifth communication flow path 145 of the second thick film sheet 140, an opening making up a flow path connecting the second communication flow path 142 of the second thick film sheet 140 and the valve 32, an opening making up a flow path connecting the third communication flow path 143 of the second thick film sheet 140 and the pressure sensor 33, and an opening making up a flow path connecting the fourth communication flow path 144 of the second thick film sheet 140 and the atmospheric release port 104.

In addition, the buffer films 250 and 260 and the double-sided tapes 270 and 280 are also provided with the openings making up the screw insertion holes 109a, 109b, 109c and 109d, and an opening making up the opening portion 108 for preventing interference with the components housed inside the main body case 11.

The thin film sheet 290 of the present modification is composed of orifice formation portions 291 and 292 made of a metal (such as nickel). In addition, a film portion 293 made of a resin (such as PET) and formed to have substantially the same thickness as that of the orifice formation portions 291 and 292 is disposed at the same layer as the thin film sheet 290. More specifically, the film portion 293 is provided with an opening portion with a shape slightly larger than the areas of the orifice formation portions 291 and 292, and the orifice formation portions 291 and 292 and the film portion 293 are disposed such that the orifice formation portions 291 and 292 are within the opening portion in plan view.

Note that a first orifice 295 is formed in the orifice formation portion 291, and a second orifice 296 is formed in the orifice formation portion 292. The orifice formation portions 291 and 292 may also be formed by electroforming, for example. By forming the orifice formation portions 291 and 292 (the thin film sheet 290) by electroforming, the orifice diameter can be accurately formed, and the manufacturing cost can be reduced by reducing the overall area of the thin film sheet 290.

Note that since the orifice formation portions 291 and 292 and the film portion 293 are separate members, the joining stability of the members and sealing of the flow path can be enhanced by providing the layers of the double-sided tapes 270 and 280. In addition, by providing the layers of the buffer films 250 and 260, constriction of the flow path in the Z direction can be suppressed.

In addition, by providing the flow path plate unit 200 in the multilayered form as in the present modification, a complex flow path can be formed.

### Others

The descriptions of the above examples are merely illustrative of the present invention, and the present invention is not limited to the specific embodiments described above. Various modifications and combinations may be made within the scope of the technical concept of the present invention. For example, in the above-described examples, the thin film sheet is provided with two orifices, but the number of orifices is not limited to two, and one orifice, or three or more orifices may be provided. That is, the flow path within the flow path plate unit may be freely designed.

In addition, in the above-described modification, the film portion 293 covers the entire region except for the thin film sheet 290 in plan view of the flow path plate unit 200 as illustrated in FIG. 10, and is formed in a shape with the opening within which the orifice formation portions 291 and 292 are accommodated, and, is disposed at the same layer as the thin film sheet 290 (the orifice formation portions 291 and 292), but this configuration is not limitative. For example, it is possible to adopt a configuration in which a plurality of small film portions 293 are dispersedly disposed on the same layer as the thin film sheet 290, or a configuration in which no film portion 293 is provided.

In addition, the fluid flowing through the flow path plate unit is not limited to a gas such as air, and the flow path plate unit may be configured as a liquid flow path. In addition, the flow path plate unit can also be used in blood pressure measurement devices other than wristwatch-type devices.

### Reference Signs List

1 . . . Blood pressure measurement device
10 . . . Main body portion
11 . . . Main body case
12 . . . Display
13a, 13b . . . Operation button
14 . . . Lug
16 . . . Cuff cover
17 . . . Control board
20 . . . Belt portion
21 . . . Belt
22 . . . Pressing cuff
24 . . . Sensing cuff
25 . . . Surface fastener
31 . . . Pump
32 . . . Valve
33 . . . Pressure sensor
91 . . . Rechargeable battery
100, 200 . . . Flow path plate unit
101 . . . Pump connecting portion
102 . . . Valve connecting portion
103 . . . Sensor connecting portion
104 . . . Atmospheric release port
106 . . . Pressing cuff connecting portion
107 . . . Sensing cuff connecting portion
108 . . . Opening portion
109a, 109b, 109c, 109d . . . Screw insertion hole
110 . . . First metal plate
111 . . . Pump connection hole
112, 132, 192 . . . Valve connection hole
113, 133, 193 . . . Sensor connection hole
114, 134 . . . Atmospheric connection hole
119a, 119b, 119c, 119d, 129a, 129b, 129c, 139a, 139b, 139c, 149a, 149b, 149c, 199a, 199b, 199c . . . Positioning opening
120 . . . Second metal plate
121a, 121b, 121c, 141a, 141b, 191a, 191b . . . Pressing cuff connection hole
122a, 122b, 122c . . . Sensing cuff connection hole
130 . . . First thick film sheet
131 . . . First communication flow path
140 . . . Second thick film sheet
142 . . . Second communication flow path
143 . . . Third communication flow path
144 . . . Fourth communication flow path
145 . . . Fifth communication flow path
190, 290 . . . Thin film sheet
195, 295 . . . First orifice
196, 296 . . . Second orifice
250, 260 . . . Buffer film
270, 280 . . . Double-sided tape
291, 292 . . . Orifice formation portion
293 . . . Film portion
T . . . Wrist

What is claimed is:

1. A flow path plate unit in which a flow path for fluid is formed, and connecting portions with a pump and a cuff are provided, the flow path plate unit comprising:
a first plate member in which at least the connecting portion with the pump is formed;
a second plate member in which at least the connecting portion with the cuff is formed;
a thin film sheet disposed between the first plate member and the second plate member and provided with one or more orifices;
a first thick film sheet disposed between the first plate member and the thin film sheet and formed to have a thickness greater than that of the thin film sheet, the first thick film sheet including a flow path through which a corresponding one of the orifices in the thin film sheet and the connecting portion with the pump in the first plate member communicate with each other;
a second thick film sheet disposed between the second plate member and the thin film sheet and formed to have a thickness greater than that of the thin film sheet, the second thick film sheet including a flow path through which a corresponding one of the orifices in the thin film sheet and the connecting portion with the cuff in the second plate member communicate with each other; and
a film member disposed between the first plate member and the second plate member, and having substantially the same thickness as that of the thin film sheet, the film member including an opening portion having a shape within which the thin film sheet is accommodated in plan view, wherein
the thin film sheet is a metal sheet in which the orifices are provided, and is formed in a shape with an area smaller than each of the first plate member, the second plate member, the first thick film sheet, and the second thick film sheet, the thin film sheet being disposed at the opening portion of the film member.

2. The flow path plate unit according to claim 1, wherein the first plate member, the first thick film sheet, the thin film sheet, the second thick film sheet, and the second plate member are joined to each other by a bonding member.

3. The flow path plate unit according to claim 2, wherein
the first thick film sheet and the second thick film sheet are double-sided tapes,
the first thick film sheet is joined to the first plate member on one surface, and joined to the thin film sheet on the other surface, and
the second thick film sheet is joined to the second plate member on one surface, and joined to the thin film sheet on the other surface.

4. The flow path plate unit according to claim 1, wherein the thin film sheet is formed by electroforming.

5. The flow path plate unit according to claim 1, wherein
the first thick film sheet and the second thick film sheet are double-sided tapes, and
the flow path plate unit further comprises:
a first film sheet disposed between the first thick film sheet and the thin film sheet,
a first bonding sheet disposed between the first film sheet and the thin film sheet,
a second film sheet disposed between the second thick film sheet and the thin film sheet, and
a second bonding sheet disposed between the second film sheet and the thin film sheet.

6. The flow path plate unit according to claim 1, wherein the orifices are formed to have a tapered shape that opens on a side into which the fluid flows.

7. The flow path plate unit according to claim 1, wherein in each of the first plate member, the first thick film sheet, the thin film sheet, the second thick film sheet, and the second plate member, a notch or a hole is formed for positioning the first plate member, the first thick film sheet, the thin film sheet, the second thick film sheet, and the second plate member when they are to be joined to each other.

8. A blood pressure measurement device comprising the flow path plate unit according to claim 1.

9. A blood pressure measurement device comprising the flow path plate unit according to claim 2.

10. A blood pressure measurement device comprising the flow path plate unit according to claim 3.

11. A blood pressure measurement device comprising the flow path plate unit according to claim 5.

12. A blood pressure measurement device comprising the flow path plate unit according to claim 6.

13. A blood pressure measurement device comprising the flow path plate unit according to claim 7.
